(19) United States
(12) Patent Application Publication
(10) Pub. No.: US 2013/0304017 A1
(43) Pub. Date: Nov. 14, 2013

(54) PEFORMANCE OF NEEDLE-FREE INJECTION ACCORDING TO KNOWN RELATIONSHIPS

(71) Applicant: Daniel E. Williamson, Sherwood, OR (US)

(72) Inventor: Daniel E. Williamson, Sherwood, OR (US)

(73) Assignee: BIOJECT, INC., Tigard, OR (US)

(21) Appl. No.: 13/467,956

(22) Filed: May 9, 2012

(51) Int. Cl. A61M 5/307

(52) U.S. Cl. 604/500; 604/70

(57) ABSTRACT

In various embodiments, an injector control system may be configured to control a needle-free injector to inject fluid injectate to a desired penetration depth. The injection control system may also be configured to receive injection performance data and use the injection performance data to make further adjustments to the needle-free injector. In various embodiments, the needle-free injector may be configured to be adjustable according to either an injection pressure and/or a nozzle orifice diameter in order to achieve injection at the desired penetration depth. In various embodiments, the needle-free injector may be configured to inject the fluid injectate according to a combined injection relationship that is both substantially linear between the adjusted pressure and the desired penetration depth and between the nozzle orifice diameter and the desired penetration depth. Other embodiments may be described and claimed.

### BACKGROUND

Needleless hypodermic injection devices are used in various situations for administration of medicines and vaccines. These devices, also known as jet injectors, typically use spring or compressed gas driven plungers to accelerate a fluid injectate, typically stored in an ampule or other reservoir, with a velocity sufficient to pierce through the skin and enter underlying tissues. Some injectors are often constructed as portable devices that may be taken to a site for administration of the injectate.

For example, FIG. 1 shows a prior art example of a needle-free injector device 100. Injector device 100 may include a body 12 to enclose various systems used to effect an injection. As illustrated in FIG. 1, body 12 may be comprised of various subsections, such as housings 14, 16, and 18. Body 12 may include an opening 22 in an end of the device 100 that may be adapted to receive a nozzle assembly 24. Nozzle assembly 24 may be configured to be selectively coupled to an injection mechanism. Nozzle assembly 24 may include an injectate chamber 26 adapted to accommodate a volume of injectate, and an outlet orifice 28 through which the injectate is ejected from device 100. Nozzle assembly 24 may further include a plunger 32 configured to move through injectate chamber 26 toward outlet orifice 28 to expel an injectate.

Injector device 100 includes one or more systems to effect an injection. For example, housed within body 12 is a drive source, such as a spring 34, disposed between spring stop members 36, 38, such that bringing spring stop members 36, 38 closer together compresses spring 34, and decompressing spring 34 pushes stop members 36, 38 away from one another. Spring stop member 38 is typically coupled to a rod 40 that may extend beyond spring stop member 36 to couple to lever 42 at attachment point 44. Injector device 100 may be armed or cocked by pivoting lever 42 at hinge 46. Pivoting lever 42 about hinge 46 results in tension on rod 40, which is transmitted to stop member 38, to move stop member 38 toward stop member 36, thereby compressing spring 34. Lever 42 is normally returned to its original position by tension on rod 40 provided by small spring like that depicted at 48. Small spring 48 is typically housed within a slotted link 50, which may be a component of stop member 38. Pivoting lever 42 about hinge 46, in addition to compressing spring 34, also may compress small spring 48 between slotted link 50 and spring stop member 52, which may be coupled to the end of rod 40. Small spring 48 typically applies sufficient force on lever 42 to return lever 42 to a home position.

Stop member 38 may be coupled to shaft member 54, which is in turn coupled to (or in contact with) plunger member 56, which is shown to be coupled to plunger 32. Shaft member 54 may make contact with plunger member 56; in other systems, shaft member 54 may be physically coupled to plunger member 56, for instance with a threaded coupling or the like. Thus, pivoting lever 42 about hinge 46 results in the compression of spring 34 and the sliding of shaft member 54 (which is shown to be coupled to stop member 38) through a channel 58 in anchor member 60. This sliding of shaft member 54 moves plunger member 56 and plunger 32 away from outlet orifice 28. In other embodiments, such as when shaft member 54 is not coupled to plunger member 56, plunger 32 may be moved away from outlet orifice 28 prior to insertion in the device 100. For instance, this may be the case when pre-filled nozzle assemblies are used.

Thus, to load device 100 with injectate, for instance in preparation for administering an injection, a user may simply place the outlet orifice 28 in contact with an injectate fluid, and pivot lever 42 about hinge 46. In various embodiments, this action will create a vacuum in injectate chamber 26, and injectate will be drawn into injectate chamber 26 via outlet orifice 28. In various embodiments, injector device 100 will remain in the cocked or armed position until actuated by a user.

In use, outlet orifice 28 may be placed in contact with or adjacent to the skin of a subject in a desired location. In the depicted embodiment, pressure exerted on latch member 66 in the direction of the nozzle assembly and the patient receiving the injection will compress spring 68 against stop member 70, releasing ball bearings from notch 64 and allowing spring 34 to propel shaft member 54, plunger member 56, and plunger 32 towards outlet orifice 28, returning them to their respective home positions as shown in FIG. 1. Plunger member 56 would expel injectate from injectate chamber 26 during this process, through output orifice 28, and into the body of the patient.

In other injector systems, rather than utilize spring-based injection, compressed gas in a reservoir may be used to drive the injectate. For example, in some systems, a poppet valve connecting to the reservoir may have a gas pressure regulation end to regulate flow from the initiator valve into the reservoir. A clamp piston may be driven forward by gas pressure from the reservoir and causes jaws to clamp onto a plunger extending into an ampule. The poppet valve may open when reservoir pressure reaches the cracking pressure of the poppet valve. Gas from the reservoir may then rush through the poppet valve into a drive chamber and force a drive piston, containing the clamp piston and jaws, forward causing the plunger to slide into an ampule. A jet of injectant may be thereby discharged from the nozzle of the ampule and penetrate through a patient's skin.

However, while certain portable needleless injectors are used, in some circumstances these devices have not achieved widespread acceptance in the medical field. Significantly, characteristics of needle-free injections may vary with various aspects of the devices and of particular administration needs. For example, injection performance may vary according to pressures exerted by the injection device, a nozzle diameter of the device, a patient's size, age and weight, the nature of the injection site, and the viscosity of the injectate.

At the same time, clinical needs may call for specific and predictable performance by a needle-free injector. For example, injections into humans are classified according to four well established tissue regions in which the injectate may be deposited. These are: intra-dermal, subcutaneous, intra-muscular, and intravenous. With intra-dermal injections, the injectate is deposited in the dermis layer. With subcutaneous injections, the injectate is deposited in the adipose tissue. With intramuscular injections, the injectate is deposited in the muscle. Intra-venous are those injections deposited directly into a vein, an injection method generally not suitable for jet injection. Each of these different layers may be found at different tissues depths, and these tissue depths may vary across parts of a body as well as from person to person.

A long standing basic difficulty with jet injection has been the complex problem of determining and controlling the depth to which an injectate is injected into tissue. The repeated failures of current systems to adequately address this problem has contributed to the lack of acceptance of a handheld and portable jet injector in the medical community.

### SUMMARY

In various embodiments, a method of controlling a needle-free injection of a pressurized fluid injectant may use a needle-free injector comprising a body terminating in a nozzle. The nozzle may include an orifice with a diameter. The method may include receiving a desired penetration depth for an injection performed with the needle-free injector. The method may also include adjusting the needle-free injector to deliver the desired injection performance. The adjusting may include adjusting the needle-free injector according to a relationship between the desired penetration depth, the diameter of the orifice and an injection pressure. The relationship may include the relationship y=M*d+K, wherein M may include a factor related to the injection pressure, and d may include the diameter of the orifice.

In various embodiments, One or more computer-readable media may be described including instructions thereon. The instructions may be configured, in response to execution by a computing device, to cause the computing device to control a needle-free injection of a pressurized fluid injectant using a needle-free injector. The injector may include a body terminating in a nozzle; the nozzle may include an orifice with a diameter. In various embodiments, the instructions may cause the computing device to control the needle-free injection through causation of the computing device to receive a desired penetration depth for an injection performed with the needle-free injector. The instructions may also cause the computing device to adjust the needle-free injector to deliver the desired injection performance. The computing device may adjust through inclusion of adjustment of the needle-free injector according to a relationship between the desired penetration depth and the diameter of the orifice and an injection pressure. The relationship may include the relationship y=M*d+K, wherein M may include a factor related to the injection pressure, and d may include the diameter of the orifice.

In various embodiments, an apparatus for controlling a needle-free injection of a pressurized fluid injectant using a needle-free injector may be described. The needle-free injector may include a body terminating in a nozzle; the nozzle may include an orifice with a diameter. In various embodiments, the apparatus may include one or more computer processors. The apparatus may also include an injector control module configured to be operated by the one or more computer processors. The injector control module may be configured to receive a desired penetration depth for an injection performed with the needle-free injector. The injector control module may also be configured to adjust the needle-free injector to deliver the desired injection performance. The injector control module may be configured to adjust including adjustment of the needle-free injector according to a relationship between the desired penetration depth and the diameter of the orifice and an injection pressure. The relationship may include the relationship y=M*d+K, wherein M may include a factor related to the injection pressure, and d may include the diameter of the orifice.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates an example sectional view of a prior art needle-free injection device.

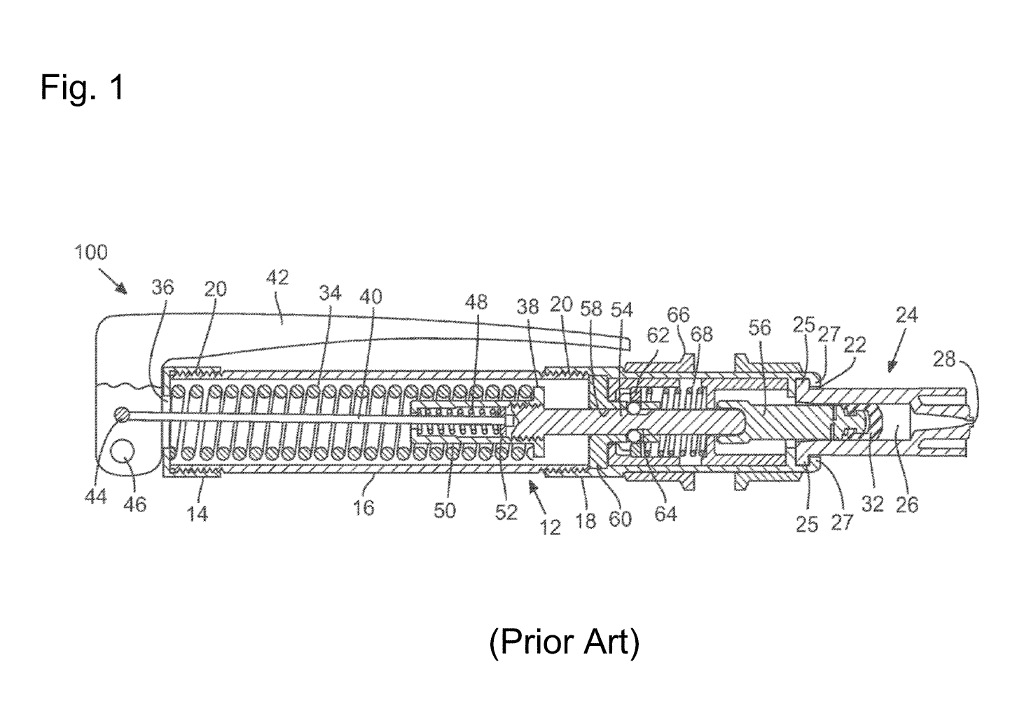

FIG. 2 is a block diagram illustrating usage of an example injector control system used to control a needle-free injector, in accordance with various embodiments.

FIG. 3 is an illustration of an example injection relationship between injection pressure, nozzle orifice diameter, and injection depth for needle-free injections, in accordance with various embodiments.

FIG. 4 illustrates an example needle-free injection and tuning process, in accordance with various embodiments.

FIG. 5 illustrates an example needle-free injector adjustment process, in accordance with various embodiments.

FIG. 6 illustrates an example needle-free injector tuning process, in accordance with various embodiments.

FIG. 7 illustrates an example computing environment suitable for practicing the disclosure, in accordance with various embodiments.

### DETAILED DESCRIPTION

In the following detailed description, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiment. Various additional operations may be performed and/or described operations may be omitted in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in various embodiments,” which may each refer to one or more of the same or different embodiments. Furthermore, the terms “comprising,” “including,” “having,” and the like, as used with respect to embodiments of the present disclosure, are synonymous.

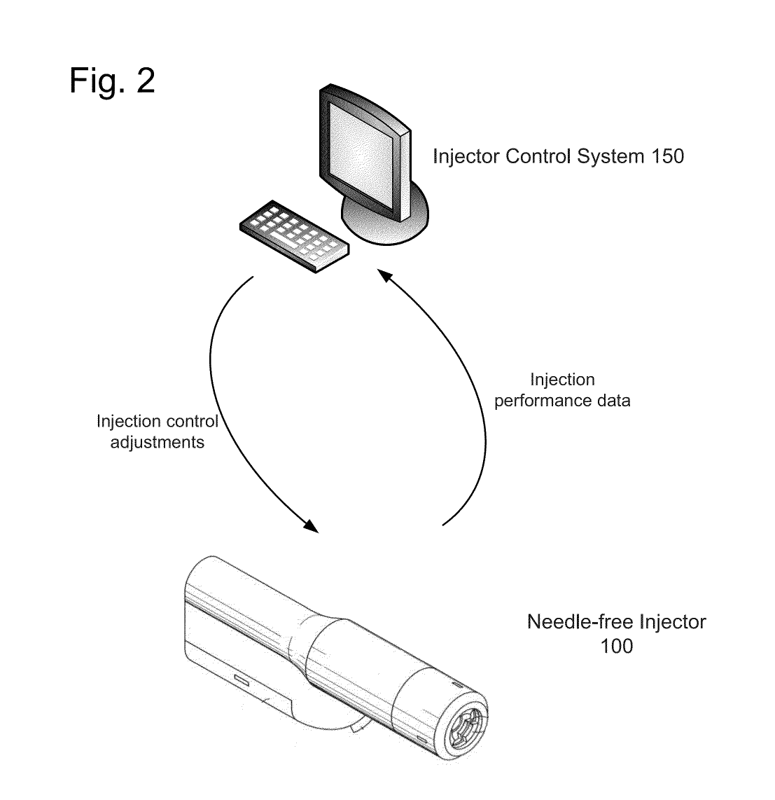

Referring to FIG. 2, an injector control system 150 is illustrated. In various embodiments, the injector control system 150 may be configured to control a needle-free injector 150 to inject fluid injectate to a desired penetration depth. In various embodiments, the injector control system 150 may be configured to send injection control adjustments to the needle-free injector 100. Various embodiments of controlling the needle-free injector 100 with the injection control adjustments are discussed below. The injection control system 150 may also be configured to receive injection performance data, such as actual penetration depth after usage of the needle-free injector. In various embodiments, the injection performance data may not be determined by the actual needle-free injector itself, but may instead be determined by another entity. The injector control system 150 may be configured to use the injection performance data to make further adjustments to the needle-free injector. In alternative embodiments, the needle-free injector itself may be configured to perform one or more features of the injector control system 150, as described below.

In various embodiments, the needle-free injector 100 may be configured to be adjustable according to either an injection pressure and/or a nozzle orifice diameter in order to achieve injection at the desired penetration depth. In various embodiments, the injector control system 100 may be configured to inject the fluid injectate according to a substantially linear relationship between the adjusted pressure and the desired penetration depth. In various embodiments, the needle-free injector 100 may be configured to inject the fluid injectate according to a substantially linear relationship between the nozzle orifice diameter and the desired penetration depth. In various embodiments, the needle-free injector 100 may be configured to inject the fluid injectate according to a combined injection relationship that is both substantially linear between the adjusted pressure and the desired penetration depth and between the nozzle orifice diameter and the desired penetration depth. In various embodiments, the needle-free injector 100 may be configured such that the relationship is adjusted according to a medium being injected, such as a gel, animal, or human tissue. In various embodiments, the injection relationship may generally describe needle-free injection performance and may be used, such as by the injector control system 150, to control injection performance during normal use. In some embodiments, the injection relationship may also be used by the injector control system 150 to analyze design for other injectors, and to predict and determine injector performance before injectors are built.

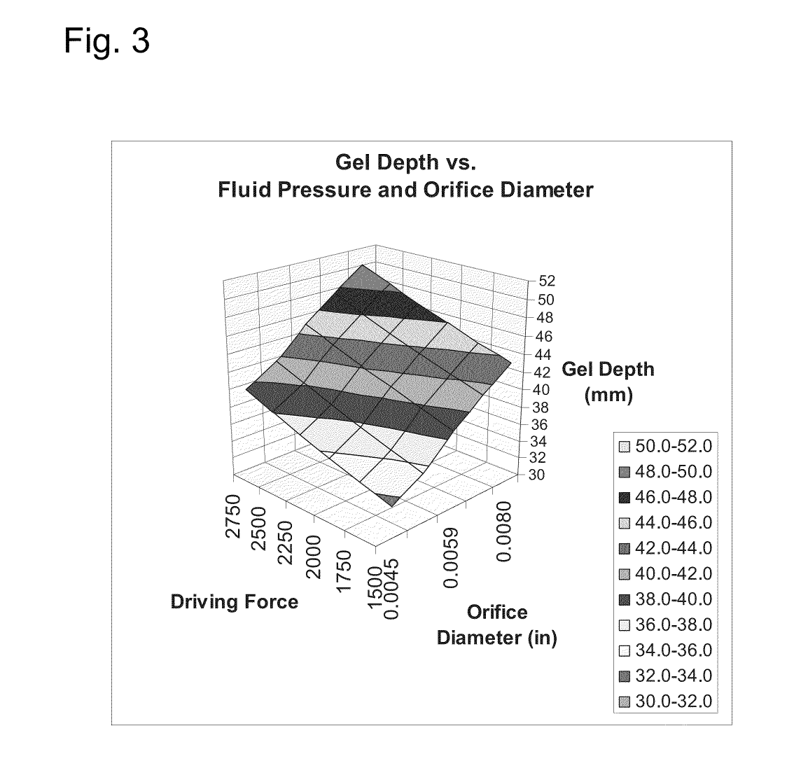

FIG. 3 is an illustration of an example injection relationship between injection pressure, nozzle orifice diameter, and injection depth for needle-free injections, in accordance with various embodiments. In various embodiments, the needle-free injector 100 may be configured such that adjustment of injection pressure and/or nozzle orifice diameter may change an expected penetration depth according to the relationship illustrated in FIG. 3.

In various embodiments, the relationship may be described by the equation y=M*d+K, where y is the penetration depth, M is a factor of injection pressure (also known as driving force, and measured in terms of force/unit area), and d is the diameter of the orifice. In various embodiments, K is a factor that relates to a medium that is being injected. In various embodiments, K may be assigned based on tissue or medium; in various embodiments, K may be based on qualities of the medium such as viscosity, resilience, or other factors that may be particular to the medium or tissue.

As the equation shows, in various embodiments, there is a substantially linear relationship between the penetration depth and the injection pressure. This may be seen in the example of FIG. 3, where a given change in the injection pressure, or “Driving Force,” along the left-to-right axis leads to the same predictable change in the penetration depth, along the vertical axis. Similarly, a given change in the orifice diameter along the front-to-back axis leads to the same predictable change in the penetration depth. In various embodiments, because the needle-free injector 100 is configured to operate based on these substantially-linear relationships, a user of the needle-free injector 100 may be facilitated in achieving a desired penetration depth for an injection through adjustment of either the pressure, the nozzle orifice diameter, or both. In various embodiments, the injection relationship may be based on the orifice cross-sectional area being directly proportional to momentum of the fluid injectate as it is streaming during injection.

As discussed above, in various embodiments, such as illustrated in FIG. 3, the relationship may be based on a particular medium or tissue being injected. For example, if the injection relationship is described as y=M*d+K, then the K factor is based on the medium being injected. In the illustrated example injection relationship, the medium being injected is an injectable gel, such as may be used for testing/tuning the injector. In other embodiments, a similar relationship may be utilized that is based on biological medium, such as human or animal tissue. Thus, penetration depths of 30-36 mm in gel may correspond to intra-dermal injection in humans, penetration depths of 36-44 mm in gel may correspond to subcutaneous injection in humans, and penetration depths in gel of >44 mm may correspond to intra-muscular injections in humans.

In various embodiments, the injection relationship may be based at least in part on placement or constitution of the injected medium or tissue. For example, women typically have a different adipose distribution than men. Men also typically have tougher tissue than women. Thus, the injection relationship being utilized by the injector may change depending on the sex of a patient. In some embodiments, a patient's age may modify the relationship. For example, infants are born with little muscle, thick layers of adipose, and very easily penetrated skin. As infants age and become mobile the adipose is gradually replaced by muscle. At adolescence the introduction of hormones changes tissue composition. Aging through mid-life is usually associated with gradual weight gain and decrease in tissue strength. Thus, the age of a patient may affect the makeup of the tissue (medium) being injected, and therefore the relationship relied upon by the injector. In other embodiments, an injection site may help determine the injection relationship, because, in various patients, skin thickness and adipose tissue may varies at different regions of the body.

In various embodiments, as the medium becomes tougher and more difficult to penetrate, the factor K may be reduced, thereby affecting penetration depth that is provided by the injector, according to the above-described relationship. In various embodiments, the effects of a particular injectable medium may be compensated for through adjustment of injection pressure and/or orifice diameter.

In various embodiments, the injector may be configured in order that the above-described relationship holds for commonly-used injection scenarios. In order that the injector 100 may perform injections in such a predictable manner, various components of the injector 100 may be configured to provide consistent injection performance.

Thus, for example, in various embodiments, a run length of the orifice of the nozzle may influence stream quality of the fluid injectate during injection. In various embodiments, the needle-free injector 100 may be configured to include a run length:orifice diameter ratio of 2.5:1 to create a collimated stream with minimal friction losses. In various embodiments, by reducing friction in the injectate stream, the needle-free injector 100 may better provide injection performance that follows the above-discussed injection relationship.

In various embodiments, run length of the nozzle orifice of the needle-free injector 100 may have an inverse effect on penetration depth of the injectate; in various embodiments, this relationship may be due to friction seen during flow of the injectate during injection. In various embodiments, fluid stream quality may also be affected by a contour of fluid path. It may be assumed, in various embodiments, that there is close to laminar flow at the orifice. The fluid stream quality may, in various embodiments, have a significant influence on momentum and velocity.

In various embodiments, the needle-free injector 100 may be configured based on an assumption that a tissue penetration is primarily related to rise time, speed, pressure and contact area. In various embodiments, it may be assumed rise time is sufficient for proper skin penetration and can be considered constant and repeatable. In various embodiments, fluid penetration may be related to cross section of the orifice (as affected by orifice diameter), pressure and the initial penetration depth.

In various embodiments, intra-dermal performance in particular may be considered as a function of fluid stream quality, an air gap (e.g., space between the orifice and skin that allows entrainment of air into the fluid stream), and a capability of patient skin to be pressurized as the injectate is added or displaces resident tissue. In various embodiments, intra-dermal injections may be performed without the use of an air gap between the nozzle orifice and skin surface if contact pressure against the skin is lessened and/or minimized. Such injections may be performed without the use of an air gap when injectate dispersion occurs before the fluid stream has a chance to penetrate dermal fascia layers separating skin from adipose tissue.

In various embodiments, the needle-free injector 100 may be configured such that fluid pressure is generated without impacting the injectate. In various embodiments, a viscosity of the injectant may also affect the ability of the injector to inject the injectate according to the relationship described above.

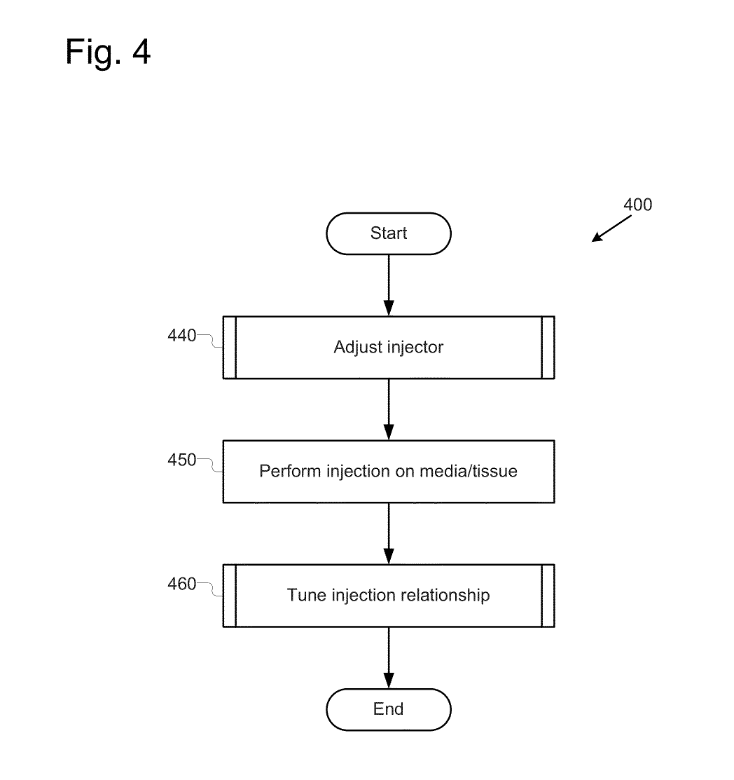

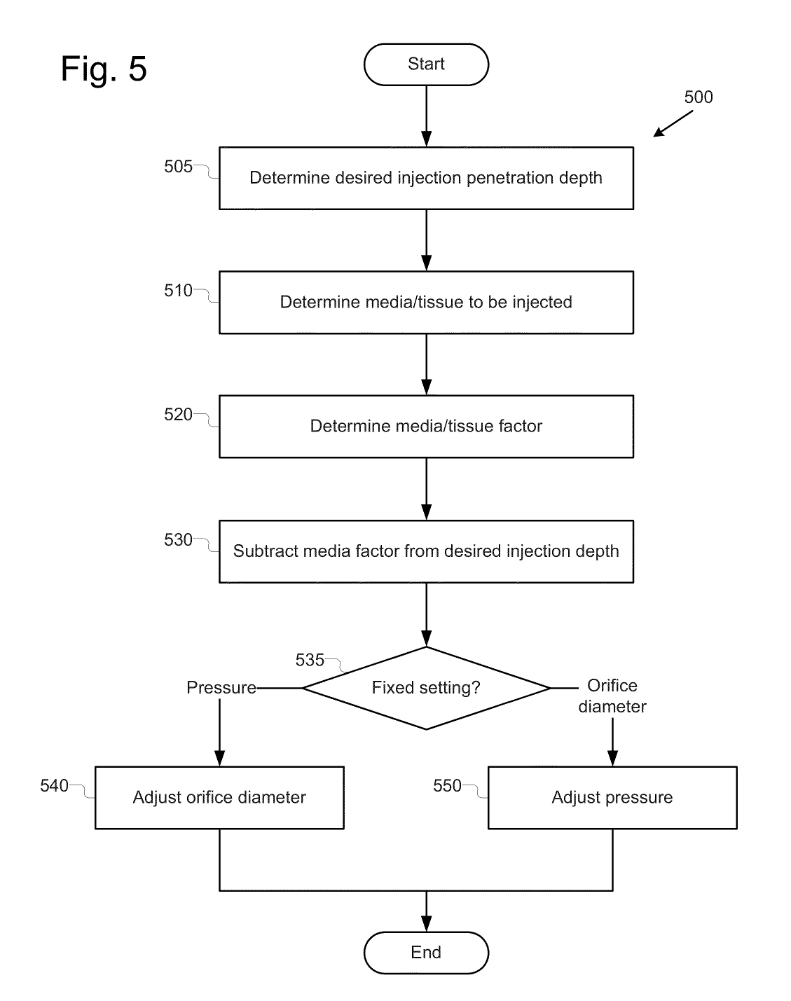

FIG. 4 illustrates an example needle-free injection and tuning process 400, in accordance with various embodiments. In various embodiments, process 400 may be performed in order to administer one or more needle-free injections according to the injection relationship described above. In various embodiments, process 400 may be performed in order to administer medicine and/or vaccines, or other medically-related fluids. In other embodiments, process 400 may be performed in order to design, analyze, and/or test performance of needle-free injectors. In various embodiments, one or more operations of process 400 (as well as various sub-processes) may be performed, in whole or in part, by the injector control system 150. The process may begin at operation 440, the needle-free injector 100 may be adjusted to perform a desired injection. In various embodiments, and as described below, operation 440 may include adjustment based on desired penetration depth and tissue or medium type. Particular embodiments of operation 440 are described below with reference to process 500 of FIG. 5.

Next, at operation 450, an injection may be performed by the needle-free injector 100 on the determined medium or tissue. In various embodiments, the injection may be performed according to an injection profile as set by the adjustments of operation 440. For example, in some embodiments, the injection may be performed by a profile that includes 1) pressurizing the fluid injectant to a peak pressure of approximately 3600-6000 psi for 3 milliseconds, 2) reducing the peak pressure to an injection pressure of approximately 1200-2000 psi for 25 milliseconds, and 3) maintaining the injection pressure adjacent to the nozzle in a substantially constant fashion until a desired dose of injectate is expelled from the nozzle.

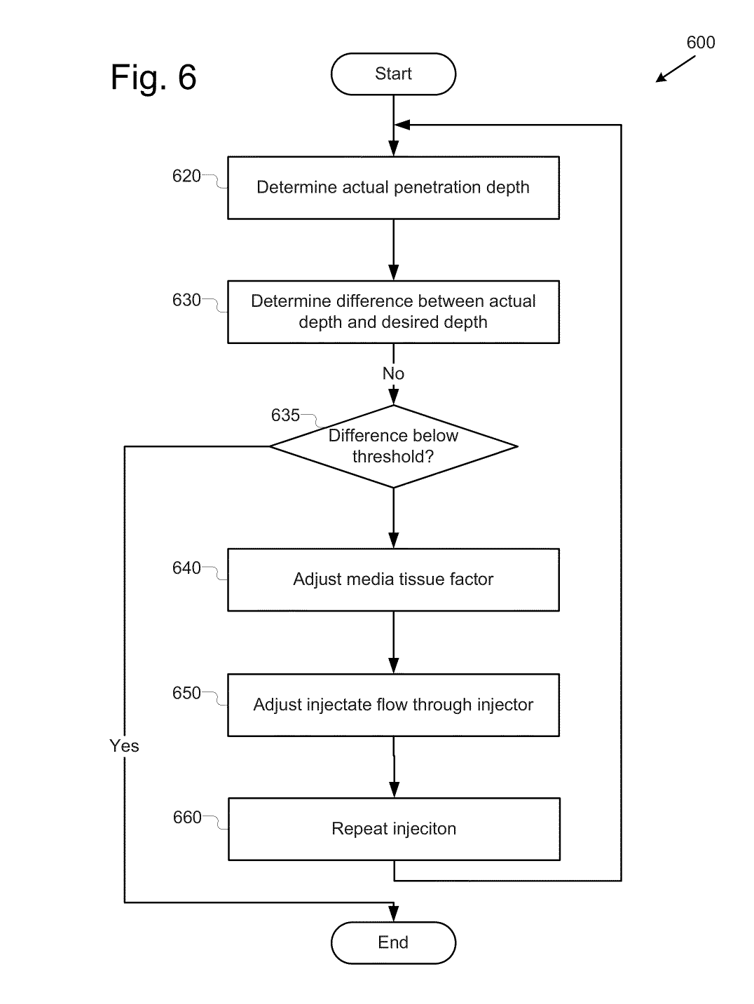

Next, at operation 460, the injection relationship may be tuned based on performance of the injection at operation 450. Particular embodiments of operation 360 are described below with reference to process 600 of FIG. 6. In various embodiments, by tuning the injector at operation 450, subsequent performance of the injector may be improved for the particular type of injection being performed. The process may then end.

FIG. 5 illustrates an example needle-free injector adjustment process, in accordance with various embodiments. In various embodiments, process 500 may include one or more embodiments of operation 440 of process 400. In various embodiments, one or more operations of process 500 may be performed, in whole or in part, by the injector control system 150. In various embodiments, the injector may be configured such that consistent injections may be performed for a given configuration of the injector. For example, in various embodiments, if injector 100 incorporates a spring, the spring type and length may be chosen to reduce ringing or other effects that would reduce injector consistency. In this manner, each injection performed by the injector, given a particular configuration of the injector, may deliver substantially similar results. In various embodiments, this consistency of injector performance may allow a user to better adjust the injector during performance of process 500.

In various embodiments, injector performance may also be tuned in real-time by adjustments to spring preload through the use of a computer-controlled piezo-electric device or other driven means of affecting spring pre-load. In other embodiments, adjustments may be made on gas-powered injectors through manipulation of input gas pressure via an electronically-controlled gas regulator.

The process may begin at operation 505, where a desired injection penetration depth may be determined. In various embodiments, and as described above, the desired penetration depth may be determined based on the type of injection desired, such as intra-dermal, subcutaneous, or intra-muscular. Next, at operation 510, a type of medium or tissue to be injected may be determined. As discussed above, this determination may include a determination that biological tissue will be injected. In various embodiments, the determination may include of one or more of species (for animal injections), patient sex, injection location, age, weight, and/or body fat percentage. In other embodiments, such as when testing is being performed, it may be determined at operation 510 that an injectable gel is being injected.

Next, at operation 520, where a factor for the medium or tissue to be injected is determined. In various embodiments, the factor may be determined based at least in part on determinations performed at operations 505 and 510.

Next, at operation 530, the medium factor is subtracted from the desired injection depth. In various embodiments, using the above described relationship y=M*d+K, this subtraction of K from y leaves the value M*d. If this value can be obtained through setting of the injection pressure and/or the diameter of the nozzle orifice, then the desired penetration depth can be achieved. In some embodiments, the value may be adjusted by holding one of the injection pressure or the diameter of the nozzle orifice constant, and then modifying the other until the desired M*d value is achieved.

Thus, at decision operation 535, it is determined if either pressure or orifice diameter is currently fixed for the injector. In various embodiments, these parameters may be fixed due to the particular configuration of the injector. Thus, the orifice diameter may not be modifiable, such as, for example, if the nozzle is not replaceable by the configuration of the injector. In such a circumstance, at operation 550, the injection pressure may be modified to achieve the desired penetration depth. In various embodiments, the injection pressure may be modified through replacement of means in the injector for providing pressure for the injection, such as replacement of a spring, adjustment of pre-load on a spring, replacement of one or more elements that provide friction during injection, or replacement of a gas reservoir. In other embodiments, the means for providing pressure may be adjusted, such as through manipulation of an adjustment means on the injector. In some embodiments as discussed above the means for providing pressure may be adjusted by adjustments to spring preload through the use of a computer-controlled piezo-electric device or other driven means of affecting spring pre-load. In other embodiments, adjustments may be made on gas-powered injectors through manipulation of input gas pressure via an electronically-controlled gas regulator.

In contrast, in some embodiments, the pressure may not be modifiable, such as for a spring-based injector with a fixed spring assembly. In such a circumstance, at operation 540, the orifice diameter may be modified to achieve the desired penetration depth. In various embodiments, the orifice diameter may be modified through replacement of the nozzle with a different nozzle having a different orifice diameter. In other embodiments, the orifice may be adjustable to modify the orifice diameter without replacement of the nozzle.

In other embodiments, if both pressure and orifice diameter are modifiable, then the injector may be modified according to one or both of these factors until the desired M*d value is achieved. In any event, once the value is achieved, the process may then end.

FIG. 6 illustrates an example needle-free injector tuning process 600 in accordance with various embodiments. In various embodiments, process 600 may include one or more embodiments of operation 460 of process 400 to tune the injector to improve its performance. In various embodiments, one or more operations of process 600 may be performed, in whole or in part, by the injector control system 150.

The process may begin at operation 620, where an actual penetration depth may be determined. In various embodiments where a gel testing medium is used this determination may be performed visually or through removal and testing of gel. In various embodiments where human or other biological tissues is used, this determination may be performed using scanning equipment, such as, for example MRI scans using a contrast medium. At operation 630, a difference between the actual penetration depth and the desired penetration depth may be determined. Next, at decision operation 635, the difference may be compared to a threshold. In various embodiments, the threshold may be set so that if the actual penetration depth is substantially similar to the desired penetration depth, the threshold is not exceeded. If the threshold is not exceed, then the process may then end.

If the threshold is exceeded, then at operations 640 and 650, various factors may be adjusted in order that the needle-free injector 100 may be better configured to operate according to the injection relationship described above. Thus, at operation 640, the medium factor may be adjusted. For example, if the actual penetration depth is smaller than expected, the medium factor may have been determined at too high of a value, and may need to be reduced. Similarly, at operation 650, the flow of the injectate through the needle-free injector 100 may be modified. For example, if the actual penetration depth is smaller than expected, then it may be determined that orifice run length is too high or that the fluid path is too contoured, providing friction that has not been accounted for.

After the adjustments of operations 640 and 650 have been performed, at operation 660, the injection may be repeated for the new adjusted factors. The process may then be repeated starting at operation 620 until the difference between the actual penetration depth and the desired penetration depth no longer exceeds the threshold. The process may then end.

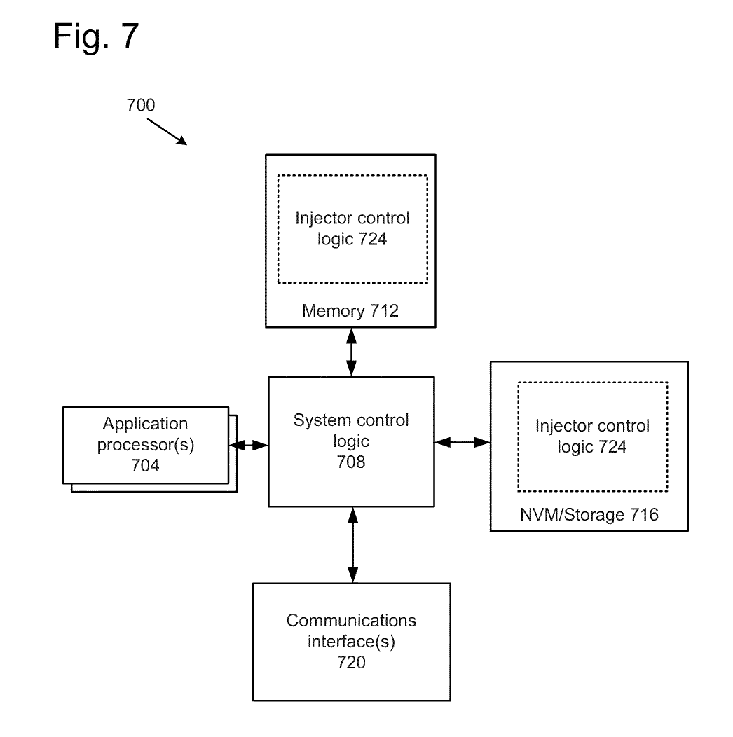

FIG. 7 illustrates, for one embodiment, an example computer system 700 suitable for practicing embodiments of the present disclosure. As illustrated, example computer system 700 may include control logic 708 coupled to at least one of the processor(s) 704, system memory 712 coupled to system control logic 708, non-volatile memory (NVM)/storage 716 coupled to system control logic 708, and one or more communications interface(s) 720 coupled to system control logic 708. In various embodiments, the one or more processors 704 may be a processor core.

System control logic 708 for one embodiment may include any suitable interface controllers to provide for any suitable interface to at least one of the processor(s) 704 and/or to any suitable device or component in communication with system control logic 708.

System control logic 708 for one embodiment may include one or more memory controller(s) to provide an interface to system memory 712. System memory 712 may be used to load and store data and/or instructions, for example, for system 700. In one embodiment, system memory 712 may include any suitable volatile memory, such as suitable dynamic random access memory (“DRAM”), for example.

System control logic 708, in one embodiment, may include one or more input/output (“I/O”) controller(s) to provide an interface to NVM/storage 716 and communications interface(s) 720.

NVM/storage 716 may be used to store data and/or instructions, for example. NVM/storage 716 may include any suitable non-volatile memory, such as flash memory, for example, and/or may include any suitable non-volatile storage device(s), such as one or more hard disk drive(s) (“HDD(s)”), one or more solid-state drive(s), one or more compact disc (“CD”) drive(s), and/or one or more digital versatile disc (“DVD”) drive(s), for example.

The NVM/storage 716 may include a storage resource physically part of a device on which the system 600 is installed or it may be accessible by, but not necessarily a part of, the device. For example, the NVM/storage 716 may be accessed over a network via the communications interface(s) 720.

System memory 712 and NVM/storage 716 may include, in particular, temporal and persistent copies of injector control logic 724. The injector control logic 724 may include instructions that when executed by at least one of the processor(s) 704 result in the system 700 practicing one or more of the injector control related operations described above. In some embodiments, the injector control logic 724 may additionally/alternatively be located in the system control logic 708.

Communications interface(s) 720 may provide an interface for system 700 to communicate over one or more network(s) and/or with any other suitable device. Communications interface(s) 720 may include any suitable hardware and/or firmware, such as a network adapter, one or more antennas, a wireless interface, and so forth. In various embodiments, communication interface(s) 720 may include an interface for system 700 to use NFC, optical communications (e.g., barcodes), BlueTooth or other similar technologies to communicate directly (e.g., without an intermediary) with another device.

For one embodiment, at least one of the processor(s) 704 may be packaged together with system control logic 708 and/or injector control logic 724. For one embodiment, at least one of the processor(s) 704 may be packaged together with system control logic 708 and/or injector control logic 724 to form a System in Package (“SiP”). For one embodiment, at least one of the processor(s) 704 may be integrated on the same die with system control logic 708 and/or injector control logic 724. For one embodiment, at least one of the processor(s) 704 may be integrated on the same die with system control logic 708 and/or injector control logic 724 to form a System on Chip (“SoC”).

Computer-readable media (including non-transitory computer-readable media), methods, systems and devices for performing the above-described techniques are illustrative examples of embodiments disclosed herein. Additionally, other devices in the above-described interactions may be configured to perform various disclosed techniques.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims.

Where the disclosure recites “a” or “a first” element or the equivalent thereof, such disclosure includes one or more such elements, neither requiring nor excluding two or more such elements. Further, ordinal indicators (e.g., first, second or third) for identified elements are used to distinguish between the elements, and do not indicate or imply a required or limited number of such elements, nor do they indicate a particular position or order of such elements unless otherwise specifically stated.

What is claimed is:

1. A method of controlling a needle-free injection of a pressurized fluid injectant using a needle-free injector comprising a body terminating in a nozzle, the nozzle having an orifice with a diameter, the method comprising:
receiving a desired penetration depth for an injection performed with the needle-free injector; and
adjusting the needle-free injector to deliver the desired injection performance, including adjusting the needle-free injector according to a relationship between the desired penetration depth and the diameter of the orifice and an injection pressure;
wherein the relationship comprises: y=M*d+K, wherein M comprises a factor related to the injection pressure, and d comprises the diameter of the orifice.

2. The method of claim 1, wherein the medium being injected comprises animal or human tissue.

3. The method of claim 1, further comprising injecting the injectate with the adjusted needle-free injector.

4. The method of claim 3, further comprising:
determining an actual penetration depth achieved after injection of the injectate; and
revising the factor related to the injection pressure and/or the factor relating to the medium being injected in the injection based on a difference between the actual penetration depth and the desired penetration depth.

5. The method of claim 3, wherein injecting the injectate with the adjusted needle-free injector comprises:
pressurizing the fluid injectant to a peak pressure for a first period of time;
reducing the peak pressure to the injection pressure over a second period of time; and
maintaining the injection pressure adjacent the nozzle substantially constant until a desired dose of injectate is expelled from the nozzle.

6. The method of claim 5, wherein:
the peak pressure comprises approximately 3600-5000 psi adjacent the nozzle;
the first time period comprises 3 milliseconds;
the injection pressure comprises approximately 1200-2000 psi adjacent the nozzle;
the second time period comprises about 25 milliseconds.

7. The method of claim 1, wherein adjusting the needle-free injector comprises, for a fixed value of the diameter of the orifice, adjusting the injection pressure to achieve the desired penetration depth.

8. The method of claim 1, wherein adjusting the needle-free injector comprises, for a fixed value of the injection pressure, adjusting the diameter of the orifice to achieve the desired penetration depth.

9. The method of claim 1, wherein:
the orifice of the needle-free injector has a run length; and
a ratio of the run length to the orifice diameter is 2.5:1.

10. One or more computer-readable media containing instructions thereon configured, in response to execution by a computing device, to cause the computing device to control a needle-free injection of a pressurized fluid injectant using a needle-free injector comprising a body terminating in a nozzle, the nozzle having an orifice with a diameter, through causation of the computing device to:
receive a desired penetration depth for an injection performed with the needle-free injector; and
adjust the needle-free injector to deliver the desired injection performance, including adjustment of the needle-free injector according to a relationship between the desired penetration depth and the diameter of the orifice and an injection pressure;
wherein the relationship comprises: y=M*d+K, wherein M comprises a factor related to the injection pressure, and d comprises the diameter of the orifice.

11. The one or more computer-readable media of claim 10, wherein the medium being injected comprises animal or human tissue.

12. The one or more computer-readable media of claim 10, wherein the instructions are further configured to cause the computing device to inject the injectate with the adjusted needle-free injector.

13. The one or more computer-readable media of claim 12, wherein the instructions are further configured to cause the computing device to:
determine an actual penetration depth achieved after injection of the injectate; and
revise the factor related to the injection pressure and/or the factor relating to the medium being injected in the injection based on a difference between the actual penetration depth and the desired penetration depth.

14. The one or more computer-readable media of claim 12, wherein the instructions are configured to cause the computing device to inject the injectate with the adjusted needle-free injector through:
pressurization of the fluid injectant to a peak pressure for a first period of time;
reduction of the peak pressure to the injection pressure over a second period of time; and
maintenance of the injection pressure adjacent the nozzle substantially constant until a desired dose of injectate is expelled from the nozzle.

15. The method of claim 14, wherein:
the peak pressure comprises approximately 3600-5000 psi adjacent the nozzle;
the first time period comprises 3 milliseconds;
the injection pressure comprises approximately 1200-2000 psi adjacent the nozzle;
the second time period comprises about 25 milliseconds.

16. The one or more computer-readable media of claim 10, wherein the instructions are configured to cause the computing device to adjust the needle-free injector through, for a fixed value of the diameter of the orifice, adjustment of the injection pressure to achieve the desired penetration depth.

17. The one or more computer-readable media of claim 10, wherein the instructions are configured to cause the computing device to adjust the needle-free injector through, for a fixed value of the injection pressure, adjustment of the diameter of the orifice to achieve the desired penetration depth.

18. The one or more computer-readable media of claim 10, wherein:
the orifice of the needle-free injector has a run length; and
a ratio of the run length to the orifice diameter is 2.5:1.

19. An apparatus for controlling a needle-free injection of a pressurized fluid injectant using a needle-free injector comprising a body terminating in a nozzle, the nozzle having an orifice with a diameter, the apparatus comprising:
one or more computer processors; and
an injector control module configured to be operated by the one or more computer processors to:
receive a desired penetration depth for an injection performed with the needle-free injector; and
adjust the needle-free injector to deliver the desired injection performance, including adjustment of the needle-free injector according to a relationship between the desired penetration depth and the diameter of the orifice and an injection pressure;
wherein the relationship comprises: y=M*d+K, wherein M comprises a factor related to the injection pressure, and d comprises the diameter of the orifice.

20. The apparatus of claim 19, wherein the injector control module is further configured to:
determine an actual penetration depth achieved after injection of the injectate; and
revise the factor related to the injection pressure and/or the factor relating to the medium being injected in the injection based on a difference between the actual penetration depth and the desired penetration depth.
